(19) United States
(12) Patent Application Publication
(10) Pub. No.: US 2025/0213463 A1
(43) Pub. Date: Jul. 3, 2025

(54) TOPICAL COMPOSITION AND USES THEREOF

(71) Applicant: COTY INC., New York, NY (US)

(72) Inventors: Olivier Doucet, Roquebrune Cap Martin (FR); Muriel Pujos, Nice (FR); Cecile Chamayou-Robert, Nice (FR)

(21) Appl. No.: 18/703,886

(22) PCT Filed: Nov. 2, 2022
(86) PCT No.: PCT/US2022/079119

§ 371 Date: Apr. 23, 2024

Related U.S. Application Data
(60) Provisional application No. 63/274,682, filed on Nov. 2, 2021.

(51) Int. Cl. A61K 8/99; A61K 8/44; A61K 8/64; A61K 31/198; A61K 35/745; A61K 38/06; A61P 17/02; A61Q 19/08

(52) U.S. Cl. CPC A61K 8/99; A61K 8/44; A61K 8/64; A61K 31/198; A61K 35/745; A61K 38/06; A61P 17/02; A61Q 19/08

(57) ABSTRACT

Provided herein are methods and compositions to improve the aesthetic appearance of skin.

### CLAIM OF PRIORITY

This patent application claims the benefit of priority to U.S. Application Ser. No. 63/274,682, filed Nov. 2, 2021, which is incorporated by reference herein in its entirety.

### FIELD

Embodiments disclosed herein include topical compositions that include a combination of creatine, palmitoylTripeptide-5 (Lys-Val-Lys) and bifida ferment lysate and uses thereof.

### BACKGROUND

The art is continually seeking new topical compositions that are storage stable, easily applied, and effective to combat a variety of disorders.

### SUMMARY

The present invention relates to topical compositions comprising creatine, palmitoylTripeptide-5 (Lys-Val-Lys) and bifida ferment lysate, and at least one carrier and uses thereof.

### BRIEF DESCRIPTION OF FIGURES

FIGURE is a bar graph depicting fibronectin levels in fibroblast cells exposed to UV irradiation and treated with creatine, palmitoylTripeptide-5 (Lys-Val-Lys) and/or bifida ferment lysate.

### DETAILED DESCRIPTION

Embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In addition, it is to be understood that the phraseology or terminology employed herein, and not otherwise defined, is for the purpose of description only and not of limitation.

All percentages and ratios used herein are by weight of the total composition and all measurements made are at 25° C., unless otherwise designated.

All numeric ranges are inclusive of narrower ranges; delineated upper and lower range limits are interchangeable to create further ranges not explicitly delineated. The number of significant digits conveys neither limitation on the indicated amounts nor on the accuracy of the measurements.

In this document, the terms “a” or “an” are used to include one or more than one and the term “or” is used to refer to a nonexclusive “or” unless otherwise indicated. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The term “topical application,” as used herein, means to apply or spread the compositions of the present invention onto the surface of the skin and also hair, nails and other mammalian, such as human, keratinous tissue.

The phrase “dermatologically-acceptable” or “cosmetically acceptable” as used herein, means that the compositions or components thereof so described are suitable for use in contact with human skin without undue toxicity, incompatibility, instability, allergic response, and the like.

The phrase “effective amount” as used herein means an amount of a compound or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound judgment of the skilled artisan.

“Skin care actives” as used herein, means substances that when applied to the skin, provide a benefit or improvement to the skin. It is to be understood that skin care actives are useful not only for application to skin, but also to hair, nails and other mammalian keratinous tissue.

“Situs” means the location where the composition is applied. Non-limiting examples of a situs include mammalian, such as human, keratinous tissue.

As used herein, the term “purified” and like terms relate to an enrichment of a molecule or compound relative to other components associated with the molecule or compound. The term “purified” does not necessarily indicate that complete purity of the particular molecule has been achieved during the process.

The term “substantially pure” describes a compound which has been separated from components which accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound is also substantially purified when it is essentially free of associated components or when it is separated from the native contaminants which accompany it in its natural state.

The terms “comprises,” “comprising,” and the like can have the meaning ascribed to them in U.S. Patent Law and can mean “includes,” “including” and the like. As used herein, “including” or “includes” or the like means including, without limitation.

### Composition

The topical compositions of the subject invention, including but not limited to lotion, serum, oil, essence, mask, night cream, stick, eye cream, color cosmetic or cream comprising a combination of creatine, palmitoyl Tripeptide-5 (Lys-Val-Lys) and Bifida ferment lysate

### Creatine

The compositions of the present invention can include creatine. Creatine is an organic compound with the formula (H₂N)(HN)CN(CH₃)CH₂CO₂H. Creatine exists in various modifications (tautomers) in solution. Creatine is found in vertebrates where it facilitates recycling of adenosine triphosphate (ATP), primarily in muscle and brain tissue. Recycling is achieved by converting adenosine diphosphate (ADP) back to ATP via donation of phosphate groups. In some aspects, creatine is present from 0.001% to 10% (w/w), including 0.002% to 9.0%, 0.003% to 8%, 0.004% to 7%, 0.02% to 5.0%, 0.05% to 4.0%, 0.10% to 3.0%, 0.20% to 2.0%, 0.04% to 1.5%, including 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.20%, 0.30%, 0.40%, 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00% 9.50% or 10.00 (w/w).

### PalmitoylTripeptide-5 (Lys-Val-Lys)

The compositions of the present invention can include palmitoylTripeptide-5 (Lys-Val-Lys). PalmitoylTripeptide-5 (Lys-Val-Lys) is a peptide and is commercially available. In some aspects, palmitoylTripeptide-5 (Lys-Val-Lys) is present from 0.000001% to 2% (w/w), including 0.000002% to 1.5%, 0.000003% to 1.0%, including 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, 0.00005%, 0.00006%, 0.00007%, 0.00008%, 0.00009%, 0.0001%, 0.0002%, 0.0003%, 0.0004%, 0.0005%, 0.0006%, 0.0007%, 0.0008%, 0.0009%, 0.0010%, 0.0020%, 0.0030%, 0.0040%, 0.0050%, 0.0060%, 0.0070%, 0.0080%, 0.0090%, 0.0100%, 0.0150% or 0.0200% (w/w).

### Bifida Ferment Lysate

The compositions of the present invention can include bifida ferment lysate. Bifida ferment lysate is a type of probiotic obtained by the fermentation of Bifida. There are commercially available sources of Bifida ferment lysate. In some aspects, Bifida ferment lysate is present from 0.0001 to 10% (w/w), including 0.0002% to 9%, 0.0003% to 8%, 0.0004% to 7%, 0.003% to 6.5%, 0.06% to 6.0%, 0.03% to 5.5%, 0.04% to 5.0%, 0.05% to 4.5, 0.06% to 4.0%, including 0.0001, 0.0002, 0.0003, 0.0004, 0.0005, 0.0006, 0.0007, 0.0008, 0.0009, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.20%, 0.30%, 0.40%, 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00% 9.50% or 10.00 (w/w).

### Carrier/Delivery System

The actives can be alone on in combination with carrier/delivery system. The compositions of the present invention comprise from about 0.000001 to about 99% of an acceptable carrier/delivery system within which the creatine, palmitoylTripeptide-5 (Lys-Val-Lys) and/or bifida ferment lysate and additional materials are incorporated to enable these components to be applied/delivered topically at an appropriate concentration. The carrier can thus act as a diluent, dispersant, solvent, or the like for the particulate material which ensures that it can be applied to and distributed evenly over the selected target at an appropriate concentration. The actives can also be encapsulated in one or more encapsulants, such as liposomes or other vehicles including biodegradable polymers/nanocapsules, hydrogels, cyclodextrins, poly(lactic-co-glycolic)acid (PLGA) or PLCA microspheres.

The carrier may contain one or more dermatologically acceptable solid, semi-solid or liquid fillers, diluents, solvents, extenders and the like. The carrier may be solid, semi-solid or liquid.

The carrier can be substantially liquid. The carrier can itself be inert or it can possess dermatological benefits of its own. Concentrations of the carrier can vary with the carrier selected and the intended concentrations of the components.

Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention. Components of the compositions of this invention should be capable of being comingled in a manner such that there is no interaction which would substantially reduce the efficacy of the composition under ordinary use situations.

The type of carrier utilized herein depends on the type of product form desired for the composition. The topical compositions useful in the subject invention may be made into a wide variety of product forms such as are known in the art. These include, but are not limited to, lotions, creams, gels, sticks, sprays, ointments, oils, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and oils.

Carriers can contain a dermatologically acceptable diluent. As used herein, “diluent” includes materials in which the material can be dispersed, dissolved, or otherwise incorporated, such as a lipophilic diluent/carrier. Solutions according to the subject invention typically include a dermatologically acceptable diluent. Solutions useful in the subject invention can contain from about 0.000001 to about 99% of the diluent.

Aerosols according to the subject invention can be formed by adding a propellant to a solution such as described above. Exemplary propellants include chloro-fluorinated lower molecular weight hydrocarbons. Additional propellants that are useful herein are described in Sagarin, Cosmetics Science and Technology, 2nd Edition, Vol. 2, pp. 443-465 (1972), incorporated herein by reference. Aerosols are typically applied to as a spray-on product.

Carriers can comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% of the dispersed hydrophobic phase and from about 1% to about 98% of the continuous hydrophilic phase; water-in-oil emulsions typically comprise from about 1% to about 98% of the dispersed hydrophilic phase and from about 1% to about 50% of the continuous hydrophobic phase. The emulsion may also comprise a gel network, such as described in “Application of Emulsion Stability Theories to Mobile and Semisolid Oil-in-Water Emulsions”, Cosmetics and Toiletries, vol. 101, November 1986, pp. 73-92, which is incorporated by reference herein.

The topical compositions of the subject invention, including but not limited to lotion, serum, oil, essence, mask, night cream, stick, eye cream, color cosmetic or cream, may comprise a dermatologically acceptable emollient. Such compositions can contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics, Science and Technology 2nd Edition, Vol. 1, pp. 32-43 (1972), incorporated herein by reference, contains numerous examples of materials suitable as an emollient.

Lotions and creams according to the present invention generally comprise a solution carrier system and one or more emollients. Lotions typically comprise from about 1% to about 20%, such from about 5% to about 10% of emollient; from about 50% to about 90%, including from about 60% to about 80%, water. A cream typically comprises from about 5% to about 50%, including from about 10% to about 20% of emollient; and from about 45% to about 85%, including from about 50% to about 75%, water.

Ointments of the present invention may comprise a simple carrier base of animal or vegetable oils or semi-solid hydrocarbons (oleaginous); absorption ointment bases which absorb water to form emulsions; or water-soluble carriers, e.g., a water-soluble solution carrier. Ointments may further comprise a thickening agent, such as described in Sagarin, Cosmetics, Science and Technology, 2nd Edition, Vol. 1, pp. 72-73 (1972), incorporated herein by reference, and/or an emollient. For example, an ointment may comprise from about 2% to about 10% of an emollient; and from about 0.1% to about 2% of a thickening agent.

Compositions of this invention useful for cleansing (“cleansers”) are formulated with a suitable carrier, e.g., as described above, and preferably contain one or more dermatologically acceptable surfactants in an amount which is safe and effective for cleansing. For example, such compositions contain from about 1% to about 90%, more preferably from about 5% to about 10%, of a dermatologically acceptable surfactant. The surfactant can be selected from anionic, cationic, nonionic, zwitterionic, amphoteric and ampholytic surfactants, as well as mixtures of these surfactants. Such surfactants are well known to those skilled in the detergency art. Nonlimiting examples of possible surfactants include isoceteth-20, sodium methyl cocoyl taurate, sodium methyl oleoyl taurate, sodium lauryl sulfate, and betaines such as described herein. See U.S. Pat. No. 4,800,197, to Kowcz et al., issued Jan. 24, 1989, which is incorporated herein by reference in its entirety, for exemplary surfactants useful herein. Examples of a broad variety of additional surfactants useful herein are described in Mccutcheon's Detergents and Emulsifiers, North American Edition (1986), published by Allured Publishing Corporation, which is incorporated herein by reference in its entirety. The cleansing compositions can optionally contain, at their art-established levels, other materials which are conventionally used in cleansing compositions.

The physical form of the cleansing compositions can be, for example, formulated as bars, liquids, shampoos, bath gels, hair conditioners, hair tonics, pastes, or mousses. Bars are most preferred since this is the form of cleansing agent most commonly used to wash the skin. Preferred rinse-off cleansing compositions, such as shampoos, include a delivery system adequate to deposit sufficient levels of actives on the skin and scalp. A delivery system can involve the use of insoluble complexes. For a more complete disclosure of such delivery systems, see U.S. Pat. No. 4,835,148, Barford et al., issued May 30, 1989, incorporated herein by reference in its entirety.

The composition may also take the form of a cosmetic composition that may be applied to mammalian keratinous tissue, including human skin. The cosmetic compositions may take various forms. For example, some non-limiting examples of forms include solutions, suspensions, lotions, oils, creams, gels, toners, sticks, pencils, ointments, pastes, foams, powders, mousses, shaving creams, wipes, strips, patches, electrically powered patches, wound dressing and adhesive bandages, hydrogels, film-forming products, facial and skin masks, cosmetics (e.g., foundations, eye liners, eye shadows), and the like.

For example, the cosmetic composition may comprise from 1% to 95% by weight of water. The cosmetic composition may comprise from 1% to 95% by weight of one or more oils. Oils may be used to solubilize, disperse, or carry materials that are not suitable for water or water soluble solvents. Suitable oils include silicones (such as dimethicone), hydrocarbons, esters, amides, ethers, and mixtures thereof. When the cosmetic composition is in the form of an emulsion, oils are carriers typically associated with the oil phase. The cosmetic composition may be in the form of a water-in-oil emulsion, an oil-in-water emulsion, or a water-in-silicone emulsion such that the cosmetic composition may include water, a silicone, oil, and combinations thereof. The cosmetic compositions may include an emulsifier. An emulsifier is particularly suitable when the cosmetic composition is in the form of an emulsion or if immiscible materials are being combined. The cosmetic composition may comprise from 0.05%, 0.1%, 0.2%, 0.3%, 0.5%, or 1% to 20%, 10%, 5%, 3%, 2%, or 1% emulsifier. Emulsifiers may be nonionic, anionic, zwitterionic, or cationic. Structuring agents may be used to increase viscosity, thicken, solidify, or provide solid or crystalline structure to the cosmetic composition. Structuring agents are typically grouped based on solubility, dispersibility, and phase compatibility. Examples of aqueous or water structuring agents include, but are not limited to, polymeric agents, natural or synthetic gums, polysaccharides, and the like. The cosmetic compositions may comprise from 0.0001%, 0.001%, 0.01%, 0.05%, 0.1%, 0.5%, 1%, 2%, 3%, 5% to 25%, 20%, 10%, 7%, 5%, 4%, or 2%, by weight of the cosmetic composition, of one or more structuring agents. The cosmetic compositions may optionally contain one or more UV actives. As used herein, “UV active” includes both sunscreen agents and physical sunblock. Suitable UV actives may be organic or inorganic. Examples of some suitable UV actives are listed in the functional category of “Sunscreen Agents” in the Personal Care Product Council's International Cosmetic Ingredient Dictionary and Handbook, Thirteenth Edition, 2010. The cosmetic compositions may be generally prepared by conventional methods such as those known in the art of making cosmetic compositions. Such methods typically involve mixing of ingredients in one or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase. The cosmetic compositions are preferably prepared such as to optimize stability (physical stability, chemical stability, photostability, etc.) and/or delivery of active materials. The cosmetic composition may be provided in a package sized to store a sufficient amount of the cosmetic composition for a treatment period. The size, shape, and design of the package may vary widely.

Optionally, the present topical composition may include one or more of the invention compositions may optionally comprise other active and inactive ingredients, including, but not limited to, excipients, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, staining agents, depigmenting agents, hypopigmenting agents, preservatives, stabilizers, pharmaceutical agents, photostabilizing agents, and mixtures thereof. In addition to the foregoing, the personal care products of the invention may contain any other compound for the treatment of skin disorders.

The cosmetic compositions disclosed herein may be applied to one or more skin surfaces and/or one or more mammalian, such as human, keratinous tissue surfaces as part of a user's daily routine or regimen. Additionally, or alternatively, the cosmetic compositions herein may be used on an “as needed” basis. In some examples, an effective amount of the cosmetic composition may be applied to the target portion of the keratinous tissue or skin. In some examples, the cosmetic composition may be provided in a package with written instructions detailing the application regimen.

### Uses Thereof

The compositions of the present invention are useful for topical application, such as a cosmetic and to improve the aesthetic appearance of skin, increased fibronectin amounts in skin and/or improve wound healing, including visible and/or tactile discontinuities in skin. Such discontinuities may be induced or caused by internal and/or external factors and include the signs of skin aging or skin wounds. The instant compositions and methods increase fibronectin in skin cells. Fibronectin is an adhesive molecule that plays a role in wound healing, particularly in extracellular matrix (ECM) formation and also in re-epithelialization. Fibronectin plays many different roles in the wound healing process because of the presence of function domains and binding sites in its structure. Fibronectin interacts with different cell types, cytokines and the ECM.

The phrase “improve the aesthetic appearance of skin” includes prophylactically and/or therapeutically improving a skin condition, including visible and/or tactile discontinuities in skin. As used herein, prophylactic use includes delaying, minimizing and/or preventing visible and/or tactile discontinuities in skin. As used herein, therapeutically improving a skin condition includes ameliorating, e.g., diminishing, minimizing and/or effacing, discontinuities in skin (improving skin appearance and/or feel).

The compositions of the present invention are useful for preventing or treating or improving signs of skin aging, more especially visible and/or tactile discontinuities in skin texture associated with aging. Improving the signs of skin aging and/or improving the aesthetic appearance of skin includes prophylactically and/or therapeutically improving one or more of such signs (similarly, improving a given sign of skin aging, e.g., lines, wrinkles or pores, includes prophylactically and/or therapeutically improving that sign). As used herein, prophylactic use includes delaying, minimizing and/or preventing signs of skin aging. As used herein, therapeutically improving such signs includes ameliorating, e.g., diminishing, minimizing and/or effacing signs of skin aging.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles, including both fine superficial wrinkles (fine lines) and coarse deep wrinkles, skin lines, crevices, bumps, large pores (e.g., associated with adnexal structures such as sweat gland ducts, sebaceous glands, or hair follicles), scaliness, flakiness and/or other forms of skin unevenness or roughness, loss of skin elasticity (loss and/or inactivation of functional skin elastin), sagging (including puffiness in the eye area and jowls), loss of skin firmness, loss of skin tightness, loss of skin density, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

It is to be understood that the present invention is not to be limited to improvement of the above mentioned “signs of skin aging” which arise due to mechanisms associated with skin aging but is intended to include improvement of said signs irrespective of the mechanism of origin. As used herein, “improving a skin condition” is intended to include improving of such signs irrespective of the mechanism of origin.

The present invention is especially useful for improving visible and/or tactile discontinuities in mammalian, such as human, skin texture, including texture discontinuities associated with skin aging. As used herein, therapeutically improving such discontinuities includes ameliorating, e.g., diminishing, minimizing and/or effacing visible and/or tactile discontinuities in the texture of mammalian, human skin, to thereby provide improved skin appearance and/or feel, e.g., a smoother, more even appearance and/or feel. Such visible and/or tactile discontinuities in skin texture include crevices, bumps, pores, fine lines, wrinkles, scales, flakes and/or other forms of textural unevenness or roughness associated with skin aging. For example, the length, depth, and/or other dimension of lines and/or wrinkles are decreased, the apparent diameter of pores decreases.

The present invention is also useful for prophylactically use that include preventing, inhibiting or delaying visible and/or tactile discontinuities in mammalian, such as human, skin texture, including texture discontinuities associated with skin aging. As used herein, prophylactic use includes delaying, minimizing and/or preventing visible and/or tactile discontinuities in the texture of mammalian skin, to thereby provide improved skin appearance and/or feel, e.g., a smoother, more even appearance and/or feel.

The compositions described herein can used to treat and/or prevent a variety of skin conditions and/or disorders. Skin conditions/disorders can include aging, signs of aging, acne, dermatitis, rosacea, psoriasis and/or wounds. Signs of aging can include wrinkles, fine lines, wizened skin, skin laxity associated with collagen loss or destruction, loss or reduction in skin integrity, lack of skin elasticity, lack of skin tone, thinned skin, sagging skin, skin suffering from degradation of collagen fibers, flaccid skin, and/or skin suffering from internal degradation. The conditions/disorders can include inflammation, broken veins, redness, blotchiness, puffy eyes or dark circles, or skin pigmentation disorders. The compositions provided herein are also useful for improving wound healing, for promoting youthful looking skin, for promoting evenness of skin tone by reducing skin redness or inflammation or UV induced skin redness, blotchiness or inflammation, for promoting skin regeneration to produce more homogenous, for promoting/increasing fibronectin levels in skin cells, firmer, more toned and more elastic skin, for promoting cell longevity, for promoting skin brightness, for promoting skin texture and tone uniformity and/or for reducing the appearance of skin pigmentation and/or skin darkening, for treating or preventing ulcerated areas or areas of cutaneous stress or damage brought about by exposure to UV or exposure to an irritant product, for enhancing skin desquamation, improving skin hydration, improving skin luster and brightness, or for treating or reducing acne or other skin blemishes.

The compositions disclosed herein may be applied to one or more topical surfaces and/or one or more mammalian, such as human, keratinous tissue surfaces as part of a user's daily routine or regimen. Additionally, or alternatively, the compositions herein may be used on an “as needed” basis and used for as intended for the given consumer product. The composition may be applied to any article, such as a textile, or any absorbent article.

Exemplary compositions of this invention include:

A composition formulated as a cream comprising:

| Ingredient | Amount (w/w of final product) |
|---|---|
| Hydrogenated polyisobutene & | |
| ethylene / propylene / styrene | |
| copolymer & butylene / | |
| ethylene/ styrene copolymer & | |
| pentaerythrithyl tetra-di-T- | |
| butyl hydroxyhydrocinnamate, | |
| dicaprylyl carbonate & | |
| tocopherol, dimethicone ethyl | |
| perfluorobutyl ether & ethyl | |
| perfluoroisobutyl ether, | |
| tocopheryl acetate | |
| Creatine | 0.001-10.00% |
| Adipocyte stem cell growth | |
| factor stimulation, essai hydro | |
| (swertia chirata extract) | |
| Alfalfa and coconut oil and | |
| squalene | |
| Cationic liposome: Water | |
| Alcohol Lecithin Retinol | |
| Polysorbate 20 | |
| Behentrimonium Chloride | |
| Potassium Phosphate Isopropyl | |
| Alcohol Tocopherol Ascorbyl | |
| Palmitate | |
| PalmitoylTripeptide 5 | 0.000001-2.00% |
| Acetyl dipeptide 1 cetyl ester | |
| Water, methylpropanediol, | |
| hyaluronic acid, silanetriol and | |
| citric acid | |
| Water; pentylene glycol, | |
| lecithin; alcohol; glycerin; | |
| xanthan gum; caprylyl glycol; | |
| propanediol; sodium hydroxide; | |
| bacillus ferment; plankton | |
| extract; hydrolyzed vegetable | |
| protein; micrococcus lysate; | |
| tetrasodium glutamate | |
| diacetate | |
| Bifida ferment lysate, sodium | 0.0001-10.00% |
| benzoate, acetic acid, lactic | |
| acid | |
| Phytocell tech malus domestica | |
| (pomme), malus Domestica | |
| fruit cell culture extract, | |
| isomalt and aqua/water | |
| Dimethylmethoxy chromanol | |
| Alpha-glucan oligosaccharide | |
| Adenosine | |
| Niacinamide | |
| Glycerin | 0.00-5% |

A composition formulated as an essence comprising:

| Ingredient | Amount |
|---|---|
| Creatine | 0.001-10.00% |
| Sodium hyaluronate | |
| Hydrolyzed hyaluronic acid | |
| Water, methylpropanediol, | |
| hyaluronic acid, silanetriol and | |
| citric acid | |
| Dimethylmethoxy chromanol | |
| Niacinamide | |
| Bifida ferment lysate, sodium | 0.0001-10.00% |
| benzoate, acetic acid, lactic | |
| acid | |
| Alpha-glucan oligosaccharide | |
| Adenosine | |
| Witch hazel distillate, alcohol, | |
| water | |

A composition formulated as an eye cream comprising:

| Ingredient | Active |
|---|---|
| Hydrogenated polyisobutene & | |
| ethylene / propylene / styrene | |
| copolymer & butylene / | |
| ethylene/ styrene copolymer & | |
| pentaerythrithyl tetra-di-T-butyl | |
| hydroxyhydrocinnamatedicaprylyl | |
| carbonate & tocopherol, | |
| dimethicone ethyl perfluorobutyl | |
| ether & ethyl perfluoroisobutyl | |
| ether, tocopheryl acetate | |
| Creatine | 0.001-10.00% |
| Adipocyte stem cell growth factor | |
| stimulation (swertia chirata | |
| extract) | |
| Alfalfa & coconut oil & squalene | |
| Cationic liposome: Water Alcohol | |
| Lecithin Retinol Polysorbate 20 | |
| Behentrimonium Chloride | |
| Potassium Phosphate Isopropyl | |
| Alcohol Tocopherol Ascorbyl | |
| Palmitate | |
| PalmitoylTripeptide-5 | 0.000001-2.00% |
| Acetyl dipeptide 1 cetyl ester | |
| Water, methylpropanediol, | |
| hyaluronic acid, silanetriol and | |
| citric acid | |
| Bifida ferment lysate | 0.0001-10.00% |
| Phytocell tech Malus domestica | |
| (pomme) or | |
| Dimethylmethoxy chromanol | |
| Niacinamide | |
| Prebiotic; alpha-glucan | |
| oligosaccharide | |
| Caffeine | |
| Horse chestnut: Phytami Marron | |
| D'inde (Aesculus Hypocastanum | |
| Seed Extract) | |
| Ginkgo Biloba Leaf Extract | |
| Adenosine | |

In one embodiment, the formulations disclosed herein can be used alone or impregnated into fibrous, cellulosic pads for application to skin. One embodiment consists essentially of creatine, palmitoylTripeptide-5 (Lys-Val-Lys) and bifida ferment lysate (in one embodiment, such a composition is impregnated into fibrous, cellulosic pads for application).

The following examples are intended to further illustrate certain embodiments of the invention and are not intended to limit the scope of the invention in any way.

### Example

Normal human primary fibroblasts were seeded in culture well plates at 200,000 cells/cm² with DMEM (Dulbecco's Modified Eagle Medium) culture medium. 24 hours later, culture medium was removed, cells were rinsed with PBS (Phosphate Buffered Saline), and fresh PBS was applied to the cells. Cells were then exposed to UV irradiation (from 300 to 400 nm) for a time to cause physical and/or chemical UV-induced change, without exposure to active ingredients. Immediately after irradiation, PBS was removed and the active ingredients creatine, palmitoylTripeptide-5 (Lys-Val-Lys) and bifida ferment lysate diluted in culture medium were applied for 72 hours. Cells were incubated at +37° C. with 5% CO₂.

Quantification of fibronectin release was performed by using Human Magnetic Luminex Assays (ref: LXSAHM-03) according to the protocol described by the supplier. Results were expressed in pg/ml.

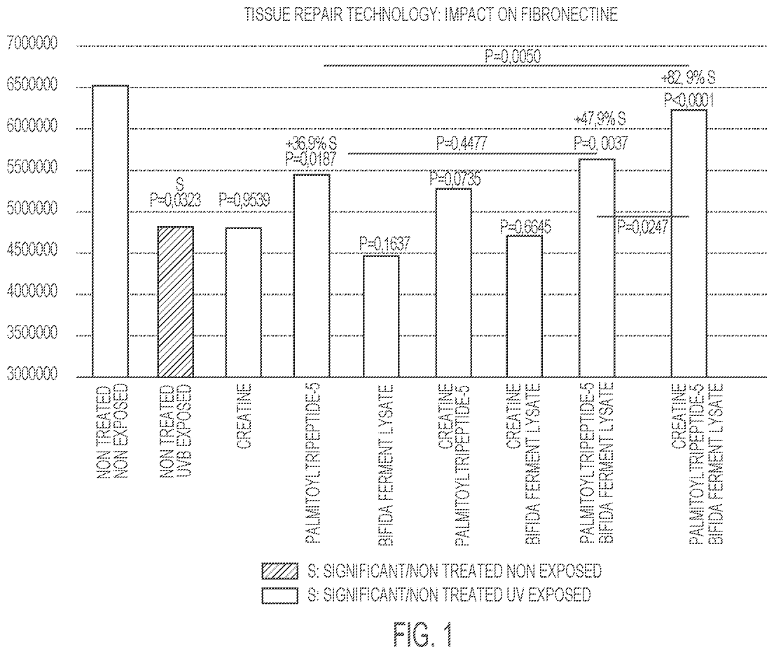

As can be seen in FIG. 1, surprisingly, a combination of palmitoylTripeptide-5 (Lys-Val-Lys) and bifida ferment lysate or a composition of creatine, palmitoylTripeptide-5 (Lys-Val-Lys) and bifida ferment lysate significantly increased fibronectin levels.

In particular, UV irradiation results in fibronectin production to decrease from 650K pg/ml to 475 μg/ml in fibroblast cells. Creatine alone does not appear to exert a positive impact on irradiated cells with regards to fibronectin production, while tripeptide-5 yields increased fibronectin production in irradiated cells to 550K pg/ml, which is a 36.9% increase over either no treatment or creatine treatment alone. Tripeptide-5 does so by specifically upregulating fibronectin production. Bifida lysate treatment demonstrates a decrease in fibronectin production in irradiated cells as compared to control cells and cells treated with creatine alone.

Creatine plus tripeptide-5 shows results similar to tripeptide-5 alone, suggesting that combining creatine with tripeptide-5 does not elevate tripeptide-5 mediated fibronectin production.

Creatine plus Bifida lysate shows a small improvement over Bifida lysate alone, but still below control. Palmitoyl tripeptide-5 plus Bifida lysate is equivalent to palmitoyl tripeptide-5 alone, suggesting no synergistic effect between palmitoyl tripeptide-5 and Bifida lysate.

However, creatine plus Bifida lysate plus tripeptide-5 shows an 82% increase in fibronectin over control, a 46% increase over tripeptide-5 alone and a 35% increase over tripeptide plus Bifida lysate. Neither creatine alone nor Bifida lysate alone were shown to have a measurable positive effect; creatine did not boost tripeptide 5's effect; and Bifida lysate only boosted tripeptide-5's performance by 11%.

While not being bound to any particular theory, creatine improves energy in cells and cells need energy for their synthesis. Bifida ferment lysate helps to improve cell repair (boosts the natural nucleotide excision repair process), which also needs energy that creatine can provide. The peptide assists in boosting fibronectin synthesis, with energy from creatine. These agents work together to boost energy and create a favorable environment for cell repair and provides the strongest boost of fibronectin.

Fibronectin is a homodimer, with two subunits of MW 250K, linked by a disulfide bridge. Fibronectin binds to receptor proteins called integrins that span the cell membrane, and also binds to extracellular matrix components such as collagen, fibrin and heparin. Fibronectin is a constituent of extracellular matrix and mediates the attachment of cells to cells and to the extracellular matrix. Fibronectin plays a role in the maintenance of skin integrity. Fibronectin, also plays a role in skin repair, is increased during the wound healing process, plays a role in cell adhesion, growth, migration, and differentiation, and it is needed for processes such as wound healing and embryonic development. Altered fibronectin expression, degradation, and organization has been associated with a number of pathologies, including aging, cancer, arthritis, and fibrosis. UV radiation increases the degradation of fibronectin in skin. In addition, fibronectin was found at decreased levels in the papillary dermis of aging skin. See Ray D. et al. J Biol Chem. 2006, 281:23060-5; Labat-Robert J. et al. J Photochem Photobiol B. 2000 57 (2-3): 113-8; Pieraggi M T et al. Ann Pathol. 1984, 4 (3): 185-94; Clark R A et al. J Invest Dermatol. 1982, 79 (5): 264-9; and Clark R A J Invest Dermatol. 1983, 81 (6): 475-9. Specifically, with age, comes downregulation of receptor to TGF-beta (Tbeta RII) associated with reduced expression of Smad3 regulated ECM components like type I Collagen and fibronectin. During aging, dermal ECM goes under fragmentation impairing fibroblasts attachment. Further, in aged skin comes a reduced size of fibroblasts accompanied by decreased production of collagen and fibronectin.

Thus, increasing fibronectin levels in skin cells will improve the appearance of aging skin, with respect to lines, wrinkles, texture, sagging, laxity and firmness of skin.

The composition and methods provided herein inhibit and treat conditions needing increased amounts of fibronectin, including aging skin and wounds.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

1. A method to improve the aesthetic appearance of skin of a human affected by aging and/or impairment of wound healing resulting from reduced fibronectin production within skin cells of the human comprising topically applying thereto a composition comprising an effective amount of creatine, palmitoylTripeptide-5 (Lys-Val-Lys) and bifida ferment lysate so as to increase fibronectin synthesis and thereby providing an anti-aging treatment and/or improved wound healing as compared to an untreated control so as to yield an improved aesthetic appearance of the human skin.

2. The method of claim 1, wherein the anti-aging is the ameliorating, treating and/or preventing fine lines and wrinkles.

3. The method of claim 1, wherein wound healing is improved.

4. A method to improve the aesthetic appearance of the skin of a human in need thereof comprising topically applying to the skin of the human at least once daily for a period of at least four weeks a composition comprising an effective amount of creatine, palmitoylTripeptide-5 (Lys-Val-Lys) and bifida ferment lysate in a cosmetically acceptable vehicle to improve the aesthetic appearance of said human's skin, wherein said skin is in need of increased fibronectin production.

5. The method of claim 4, wherein the improved the aesthetic appearance is due to the ameliorating, treating and/or preventing fine lines and wrinkles.

6-7. (canceled)

8. The method of claim 1, wherein the concentration of creatine is from 0.001% to 10% (w/w), the concentration of palmitoylTripeptide-5 (Lys-Val-Lys) is from 0.000001 to 2% (w/w) and the concentration of Bifida ferment lysate is from 0.0001 to 10%.

9. The method of claim 1, wherein the composition is formulated as a lotion, cream, serum, essence, mask, night cream, stick, eye cream or color cosmetic (such as foundation or concealer).

10. A composition formulated as a cream comprising:

| | Amount (w/w of final |
|---|---|
| Ingredient | product) |
| Hydrogenated polyisobutene | |
| & ethylene / propylene / | |
| styrene copolymer & | |
| butylene / ethylene/ styrene | |
| copolymer & | |
| pentaerythrithyl tetra-di-T- | |
| butyl | |
| hydroxyhydrocinnamate, | |
| dicaprylyl carbonate & | |
| tocopherol, dimethicone | |
| ethyl perfluorobutyl ether & | |
| ethyl perfluoroisobutyl ether, | |
| tocopheryl acetate | |
| Creatine | 0.001-10.00% |
| Adipocyte stem cell growth | |
| factor stimulation, essai | |
| hydro (swertia chirata | |
| extract) | |
| Alfalfa and coconut oil and | |
| squalene | |
| Cationic liposome: Water | |
| Alcohol Lecithin Retinol | |
| Polysorbate 20 | |
| Behentrimonium Chloride | |
| Potassium Phosphate | |
| Isopropyl Alcohol Tocopherol | |
| Ascorbyl Palmitate | |
| PalmitoylTripeptide 5 | 0.000001-2.00% |
| Acetyl dipeptide 1 cetyl ester | |
| Water, methylpropanediol, | |
| hyaluronic acid, silanetriol | |
| and citric acid | |
| Water; pentylene glycol, | |
| lecithin; alcohol; glycerin; | |
| xanthan gum; caprylyl glycol; | |
| propanediol; sodium | |
| hydroxide; bacillus ferment; | |
| plankton extract; hydrolyzed | |
| vegetable protein; | |
| micrococcus lysate; | |
| tetrasodium glutamate | |
| diacetate | |
| Bifida ferment lysate, sodium | 0.0001-10.00% |
| benzoate, acetic acid, lactic | |
| acid | |
| Phytocell tech malus | |
| domestica (pomme), malus | |
| Domestica fruit cell culture | |
| extract, isomalt and | |
| aqua/water | |
| Dimethylmethoxy chromanol | |
| Alpha-glucan oligosaccharide | |
| Adenosine | |
| Niacinamide | |
| Glycerin | 0.00-5% |

11. A composition formulated as an essence comprising:

| Ingredient | Amount |
|---|---|
| Creatine | 0.001-10.00% |
| Sodium hyaluronate | |
| Hydrolyzed hyaluronic acid | |
| Water, methylpropanediol, | |
| hyaluronic acid, silanetriol | |
| and citric acid | |
| Dimethylmethoxy chromanol | |
| Niacinamide | |
| Bifida ferment lysate, sodium | 0.0001-10.00% |
| benzoate, acetic acid, lactic | |
| acid | |
| Alpha-glucan oligosaccharide | |
| Adenosine | |
| Witch hazel distillate, | |
| alcohol, water | |

12. A composition formulated as an eye cream comprising:

| Ingredient | Active |
|---|---|
| Hydrogenated polyisobutene & | |
| ethylene / propylene / styrene | |
| copolymer & butylene / | |
| ethylene/ styrene copolymer & | |
| pentaerythrithyl tetra-di-T-butyl | |
| hydroxyhydrocinnamatedicaprylyl | |
| carbonate & tocopherol, | |
| dimethicone ethyl perfluorobutyl | |
| ether & ethyl perfluoroisobutyl | |
| ether, tocopheryl acetate | |
| Creatine | 0.001-10.00% |
| Adipocyte stem cell growth factor | |
| stimulation (swertia chirata | |
| extract) | |
| Alfalfa & coconut oil & squalene | |
| Cationic liposome: Water Alcohol | |
| Lecithin Retinol Polysorbate 20 | |
| Behentrimonium Chloride | |
| Potassium Phosphate Isopropyl | |
| Alcohol Tocopherol Ascorbyl | |
| Palmitate | |
| PalmitoylTripeptide-5 | 0.000001-2.00% |
| Acetyl dipeptide 1 cetyl ester | |
| Water, methylpropanediol, | |
| hyaluronic acid, silanetriol and | |
| citric acid | |
| Bifida ferment lysate | 0.0001-10.00% |
| Phytocell tech Malus domestica | |
| (pomme) or | |
| Dimethylmethoxy chromanol | |
| Niacinamide | |
| Prebiotic; alpha-glucan | |
| oligosaccharide | |
| Caffeine | |
| Horse chestnut: Phytami Marron | |
| D'inde (Aesculus Hypocastanum | |
| Seed Extract) | |
| Ginkgo Biloba Leaf Extract | |
| Adenosine | |
